(19) United States
(12) Patent Application Publication
(10) Pub. No.: US 2004/0146545 A1
(43) Pub. Date: Jul. 29, 2004

(54) Method for making products used in sequential micronutrition and uses of resulting products

(76) Inventor: Elisabeth Caenazzo, Cachan (FR)

Correspondence Address: JAMES C. LYDON, 100 DAINGERFIELD ROAD, SUITE 100, ALEXANDRIA, VA, 22314, US

(21) Appl. No.: 10/756,516

(22) Filed: Jan. 14, 2004

Related U.S. Application Data
(63) Continuation of application No. 09/979,126, filed on Nov. 19, 2001, now abandoned.
Application No. 09/979,126 is a 371 of international application No. PCT/FR00/01371, filed on May 19, 2000.

(30) Foreign Application Priority Data
May 19, 1999 (FR) 99/06750

(51) Int. Cl. A61K 35/78; A61K 47/00

(52) U.S. Cl. 424/439; 424/600; 424/725

(57) ABSTRACT

A method for making products used in micronutrition which includes selecting for at least two among the following types: trace element, plant, food principle, microorganism, vitamin, one or several active substances of each type depending on the functions required for the system; sequentially combining the active substances of each type by introducing sequentially each of them in an intermediate container in the form of a liquid solution, so as to obtain a partial solution; and combining sequentially and in the following order: trace element, plant, food principle, microorganism, vitamin, the resulting partial solutions by introducing sequentially each partial solution into a final container.

### TECHNICAL FIELD

This invention relates to micronutrition in which active substances are absorbed in infinitesimally small amounts in order to provide all parts of the body with the correct quantities of the factors that they need, and concerns in particular a manufacturing method for products based on active substances used in micronutrition.

### BACKGROUND ART

It is well known that nutrients such as trace elements, plants, essential dietary factors (e.g. amino acids) and vitamins may stimulate or inhibit various physiological functions, or they may be neutral, representing a simple source of energy.

Nutrients have their effect at a final destination which is the cell in the targeted part of the body, following a chain of causes and effects in which the amount of the nutrient present (which is difficult to measure) may be either too great or insufficient (in which case, supplementation is necessary). One of the features of conventional weight-biased nutrition is to provide sufficiently large quantities to insure the correction of any deficiencies. Unfortunately, when certain nutrients are provided in very high quantities, they can block the sites at which other nutrients are absorbed.

That the absorption of certain nutrients can be blocked in this way is shown by the fact that, some years ago, a recommendation against combining calcium and magnesium was issued. The reason given was that, since both elements are absorbed at the same sites, one of them will no longer be efficiently absorbed if they are administered at the same time. However, it is now known that, as long as calcium intake is below 800 mg per day, magnesium can be absorbed simultaneously. This is an example of competition for intestinal absorption sites, a question for which conventional weight-biased nutrition fails to provide a satisfactory answer. But there are other antagonistic nutrients which cannot be provided simultaneously to the body in the form of weight-biased nutrition. For example, such is the case for certain pairs of a metal and another trace element: molybdenum and phosphorus, iron and phosphorus, manganese and magnesium, copper and iron, copper and zinc; or metal-amino acid pairs like arginine and zinc; or different amino acids like isoleucine which competes with tyrosine, tryptophan and phenylalanine.

Micronutrition consists in providing the body with very small amounts of one or more nutrients in order to resolve some of the problems associated with conventional weight-biased nutrition. The smallness of the quantities means that the nutrients can be directly assimilated at the final destination (i.e. the cell) which is not the case with weight-biased nutrition. However, most importantly, micronutrition overcomes problems associated with the saturation of absorption sites. Micronutrition does not result in the saturation of intestinal absorption sites.

Unfortunately, micronutrition alone does not allow a sufficiently precise manner in which to target any particular physiological function or organ since it does not itself constitute a structure which can be readily recognized and therefore selectively absorbed at the target. In practice, micronutrition alone, administered to and absorbed by the body in a global fashion, compensates sub-deficiencies but is not enough to compensate deficiencies.

### DISCLOSURES OF THE INVENTION

This is why the purpose of the invention is to develop a micronutrition modality in which the active substances used (such as nutrients or active substances administered for therapeutic ends) are administered in a precise order so that they are recognized at the target, thereby resulting in potentiation of the effect of each of the active substances included in the sequence.

This purpose is achieved by implementing an original manufacturing method for the micronutritional product which enables this potentiation for each of the active substances in the selected sequence.

According to a first embodiment, the method consists in selecting, based on the physiological effects sought, one or more active substances from at least two of the following groups: “trace element”, “plant”, “essential dietary factor”, “microorganism”, and “vitamin”, in combining the active substances of each group by introducing each sequentially in the form of a solution in the correct order into an intermediate receptacle so as to obtain a partial solution, and in combining sequentially, according to the pre-established order of the groups: “trace element”, “plant”, “essential dietary factor”, “microorganisms”, and “vitamin”, the partial solutions obtained by sequentially introducing each partial solution into a final receptacle after having dynamized the solution containing the partial solution(s) already contained in the final receptacle by shaking.

According to a second embodiment of the invention, the stage consisting in sequentially combining the partial solutions is replaced by a stage consisting in coating a plurality of granules by spraying droplets of each of the partial solutions in the pre-established order of the “groups-trace element”, “plant”, “essential dietary factor”, “microorganism”, and “vitamins”, so as to obtain a final product in the form of granules carrying a series of successive layers formed according to the pre-established sequence.

### DESCRIPTION OF THE INVENTION

Firstly, the active substances used belong to the groups: “trace element”, “plant”, “essential dietary factor”, “microorganism”, and “vitamin”.

The “trace elements” are metallic or metalloid elements from the Periodic Table which are found in the body.

The “plants” are, as a general rule, those plants which have phytotherapeutic activity, also referred to as “medicinal plants”. They may come from Europe, Africa, the Amazon Forest, Madagascar or North Africa, or any plant of this type with scientifically recognized or traditionally ascribed effects.

The “essential dietary factors” may be vegetables, fruit, seaweed, microscopic mushrooms or, in a general way, any dietary substance which acts as a source of amino acids for the body.

The “microorganisms” may be yeast organisms, other microscopic fungi or bacteria containing substances with nutritional or therapeutic value.

The “vitamins” may be any substance which is considered to be a vitamin in physiological terms.

One of the invention's essential characteristics therefore is the blending of the selected active substances in the order of the groups: “trace element”, “plant”, “essential dietary factor”, “microorganism”, and “vitamin”. In other words, an active substance belonging to a given group is only introduced into the product to be made after the introduction of the active substance(s) belonging to the preceding group in the list above. However, it is not indispensable that active substances from every group should be present in the product to be made. Thus, a product could be imagined which only contains active substances from two or three of the five groups stipulated without departing from the framework of the invention. For information, the number of possible active substances in a micronutritional product is unlimited and is usually between 10 and 40.

Another essential characteristic of the invention is the introduction into the product to be made of active substances from a given group in a pre-established, sequential order. Thus, for a given function such as immune function, trace elements might be introduced in the order magnesium, then iron and then zinc, whereas for detoxication function, the order would be zinc, then magnesium and finally iron.

Assuming that the final product is to be a combination of active substances from all five groups, the manufacturing method would consist in putting the first active substance (in aqueous or alcoholic solution) from the “trace elements” group into the intermediate receptacle, then dynamizing the solution by shaking it before introducing the second active substance from the “trace element” group. The solution is again dynamized by shaking prior to the introduction of the third active substance (if there is one), and so on until all the active substances belonging to the “trace element” group have been added. Finally, the mixture is dynamized by shaking so as to obtain a partial solution.

In a separate intermediate receptacle, the same manipulations are performed with the active substances from the “plant” group to generate a new, partial solution after dynamization following the addition of the final “plant” active substance.

The same manipulations are performed with active substances from the “essential dietary factor” group to generate a third partial solution in a third intermediate receptacle.

The same manipulations are performed with the active substances from the “microorganism” group to generate a fourth partial solution in a fourth intermediate receptacle, and with the active substances from the “vitamin” group to generate a fifth partial solution in a fifth intermediate receptacle.

The final phase then consists in combining the partial solutions contained in the intermediate receptacles by adding each solution sequentially in the correct order of “trace element”, “plant”, “essential dietary factor”, “microorganism”, and “vitamin”, after having dynamized the mixture in the final receptacle by shaking.

It should be noted that, although the method according to the invention has been described above using five groups of active substances, it is clear that the same method can be applied using just two, three, four or five of the groups, as long as they are added in the order stipulated. Moreover, although dynamization by shaking might be preferable, the method can be implemented without the dynamization step.

For information, dynamization by shaking induces motion at the molecular level. The technique consists in shaking the receptacle containing the solution to be dynamized one hundred times without interruption.

As a general rule, the same quantity of each of the partial solutions is introduced into the final receptacle. Thus, if all five groups are used, the proportions will be: ⅕ for the “trace element” group; ⅕ for the “plant” group; ⅕ for the “essential dietary factor” group; ⅕ for the “microorganism” group; and ⅕ for the “vitamin” group.

The quantity of each active substance can vary as long as it is kept small. The quantity used is preferably between 10⁻² g and 10⁻⁹ g per liter of solution. It should be noted that the solutions of the active substances belonging to the “plant”, “essential dietary factor” and “microorganism” groups are prepared from stock tinctures. Preparations obtained by the method described above can be used directly in the form of a liquid or in a gel.

Micronutritional products can also be used in a solid form in tablets or capsules, or added to salt. In this case, use is made of granules made of either lactose or of a mixture of lactose and sucrose which have been processed in a turbine. A compressor is used to spray droplets of a solution of the active substance in such a way as to deposit the active substance on the surface of the granule. The active substances are sprayed onto the granules in the same sequence—with respect to both the various groups and the different active substances belonging to each of the groups—as that used for liquid preparations, thus forming successive layers of the various active substances after evaporation.

Active substances from each group are selected on the basis of the desired result. One of the parameters used to make this selection could be electromagnetic field measurements. For this, throughout the production process regular checks are made of the electromagnetic field of the system: function or organ.

The selection made may affect the targeted function, how it is controlled upstream, or what it controls downstream. The selection of active substances is made in an order in which it is checked that each selected substance potentiates the action of the preceding ones and makes it possible to obtain an effect, i.e. a more positive response at the target than if the active substance were not present. This assessment of efficacy is made from mutually complementary parameters and observations. In this way, a greater effect is obtained at the target without any negative impact or decompensation either upstream, downstream or in peripheral systems.

In the final analysis, the selection of substances necessitates defining and investigating those which act to repair the targeted part of the body which is compromised or weakened by observing the phenomena of deficiency related to the situation, and in parallel, testing all possible combinations of different active substances until the best clinical outcome is obtained.

The products generated by the sequential manufacturing methods described above make it possible to induce particularly potent, accurately targeted effects, on the one hand because the sequential method guarantees potentiation of the effectiveness of each of the substances used with respect to the objective for which they were selected and, on the other hand, because the method results in precise action on the targeted organ or function since the sequence is selected for its capacity to focus the effect of the action at the target.

Sequenced micronutrition makes it possible to have well-differentiated nutritional action according to the objectives set. This is unique in its mode of production and its mode of functioning. Apart from potentiating the action of each of the substances, it makes it possible to put all of the different substances in a synergistic relationship thus extending the action of each one as well as enhancing and potentiating the overall effect.

The result of sequenced micronutrition is to strengthen organs and restore functionality so that they can be active in the individual's constant processes of adaptation to the environment and changing situations.

The effects of sequenced micronutrition will help the defense capabilities of organs and their function when the body is weak or under attack. This support will help the organ or function to respond better by restoring its capacity for self-regulation.

As previously mentioned, the products of sequential micronutrition can be directly assimilated at the final destination (the cell) thereby avoiding slow, energy-consuming conversion processes which necessarily generate waste materials, and they also make it possible to blend a large number of active substances from different, complementary sources which cannot be combined in weight-biased preparations.

As a result, they give comprehensive, far-reaching nutritional action which will restore a ground and replenish the whole body.

Applications for the Micronutritional Products Obtained

1. A major set of applications in humans and animals concerns self-regulation, general nutrition, therapy (for pathology and general health) and the re-establishment of all types of physiological functions:

detoxication and toxin removal: clearance, drainage and cleansing functions and all its applications in all chronic functional and organic problems, in acute and chronic drug poisoning, in various forms of pollution, in quitting smoking, in drug and alcohol severance, in detoxication following general anesthesia or repeated local anesthesia, and in the sequelae of immunization.

General muscle tone which affects overall vitality and all its applications: countering tiredness, depression, the resistance to exertion in athletes, recovery from disease, from a surgical procedure, muscle tone in the elderly and in pregnant women.

The circulatory systems of the body and its applications in the venous circulatory system: venous insufficiency, arterial circulation: atheroscelerosis, cerebral circulation: cerebral insufficiency in the elderly, stroke syndrome; the cellular and general circulation, osteopathic care modalities.

Gastrointestinal function as a whole, gastric function: dyspepsia, post-prandial heaviness; liver function: impaired liver function or ongoing hepatic poisoning or its sequelae: virally mediated or induced by alcohol or drugs; pancreatic function: pancreatic insufficiency, pancreatitis, diabetes; bladder function: spasm; intestinal function: problems with passage: constipation, diarrhea, spasmodic colitis; migraine.

The function of cellular vitality and its applications in the defense against free radicals, the mitigation of the aging process, recovery of cellular vitality after massive cell loss: radiotherapy, chemotherapy, viral hepatitis, thalassemia; endocrine insufficiency.

Sleep function and its applications in problems with going to sleep, nocturnal waking, early waking.

Skeletal function, prevention, maintenance and self-regulation of bone tissue and its applications in: osteoporosis, consolidation after fracture, growth.

Visual function, visual fatigue with prolonged concentration: driving, working at a computer.

Memory function and concentration: its applications before and during an examination, in the elderly, mental strain.

Stress regulation function, all forms of stress and their decompensation: emotional shock, physical injury, transient or chronic strain.

Cerebral function and its applications: in the elderly, in all forms of cerebral fatigue: strain in dyslexia and in cerebral stress of all degrees: degeneration, anoxia, altitude sickness, skull injury.

2. The prevention, care, re-equilibration and re-establishment of the following organs:

the rectum, uterus, ovaries, prostate, testicles, kidneys, bladder, colon, small intestine, adrenal glands, spleen, thymus, pancreas, bile duct, liver, stomach, lymphatic system, arteries, cardiac veins, breasts, lungs, larynx, rhinopharynx, ears, eyes, sinus, brain, and the vertebrae: sacral, lumbar, cervical and the cranial bones.

3. Nutrition and regulation of specific points of the body:

Application in Chinese medicine: acupuncture points, reflexology, shiatsu-type massage

4. Local regeneration and revitalization and its applications: in pain, contusions, burns, bites of all sorts, cutaneous symptoms: eruptions, infectious complications, localized joint pain, local collapse.

5. Metabolic regulation

6. Specific vitalization of the hair

7. Specific vitalization of the skin

8. Weight regulation and loss:

Re-education of the sense of taste, fluid balance, appetite control, regulation of the metabolic processes which control the assimilation of sugar and fat.

9. Cosmetic skin care:

a) internal care: toxin removal from the skin, skin vitality, the nutritional balance of the skin, relaxation of the skin, overall skin beauty.

b) External care: detoxication of the skin, skin tone, regeneration of the skin, skin flexibility, the overall balance of the skin.

10. Bone regeneration in dentistry and its applications:

Loose and unstable teeth, the sequelae of extraction, prostheses.

11. Nutrition and revitalization of the gums.

12. Specific detoxication in dental care.

Following anesthesia, the elimination heavy metals, regeneration of collapsed structures and injuries in the mouth region.

### EXAMPLES

The examples which follow are given for illustrative purposes and are in no way to restrict the scope of the invention. Together with other information, each example provides a sequential list of the active substances of each group used with the groups listed sequentially according to the principles of the invention.

### Example 1

### Immunity Function

Quantities: active substances dosed at 4 10⁻⁸ g

Trace elements: magnesium, iron, zinc

Plants: thymus vulgaris (thyme)

Essential dietary factors: cicer arietinum (split peas), ervum lens (lentils), ribes nigrum oleum acini (blackcurrant sedd oil), elasis guinensis (palm oil), fucus vesiculosis, undaria pinatifida, palmaria palmata

Vitamins: A, B1, B2, B5, B6, B9, C, E, F, PP

Form of the product: capsules

### Example 2

### Detoxication Function

Quantities: active substances dosed at 4 10⁻⁸ g

Trace elements: zinc, magnesium, iron

Plants: anthemis nobilis (roman chamomile), tilia europea (lime), mentha veridis (mint), hibiscus abelmoschus Essential dietary factors: daucus carot (carot), nasturtum officinalis (cress), brassica aleracea (cabbage), elasis quinensis (palm oil), pirus malus (apple), amygdalus persica (peach), apium sativus (celery), cichorium intybus (chicory), chondrus crispus, himanthalia elongata (enriched yeast)

Vitamins: A, B1, B6, B9, C, E, F, H, PP

Form of the product: capsules

### Example 3

### Digestion Function

Quantities: active substances dosed at 4 10⁻⁸g

Trace elements: zinc lactate, magnesium lactate, calcium lactate

Plants: rosa canina (rose hip), verbena officinalis (vervein), hibiscus abelmoschus

Essential dietary factors: zea mays (corn), coryllus avellana (hazelnut), solanum lycopersicum (tomato), daucus carot (carot), apium sativus (celery), oenothera biennis oleum (evening primrose oil), fucus vesiculosus, ostrea (oyster), yeast enriched with selenium, simple yeast

Vitamins: A, B1, B2 Riboflavine, B3=PP, B5, B6, C, E, H, Biotine, B8

Form of the product: capsules

### Example 4

### Sleep Function

Quantities: active substances dosed at 4 10⁻⁸ g

Trace elements: magnesium lactate, calcium lactate, natrum muriaticum

Plants: citrus aurantium (orange tree), verbena officinalis (vervein), hibiscus abelmoschus, anthemis nobilis (chamomile), tilia europea (lime)

Essential dietary factors: pirus malus (apple), lactusa sativa (lettuce), prunus armeniaca (apricot), fragaria vesca (strawberry), cucumis saticus (cucumber), cucurbita pepo (pumpkin), agaricus campester (cultivated psalliota mushroom), nasturtium officinale (cress), coryulus avclana (hazelnut), chondrus crispus, ostrea (oyster)

Vitamins: A, C, E, H, PP

Form of the product: capsules

### Example 5

### Bone Function

Dosage: active substances dosed at 4 10⁻⁸ g

Trace elements: magnesium, iron, calcium, sodium

Essential dietary factors: polygonum fagopyrum (buckwheat), brassica napus (rutabaga), avena sativa (oat), castenea vulgaris (chessnut), apium sativum (celery), agaricus campester (cultivated psalliota mushroom), phoenix dactylifera (date), fucus vesiculosus, porphyra umbilicalis, ostrea (oyster)

Vitamins: A, C, E, H Biotine, PP

Form of the product: capsules

### Example 6

### Cerebral Function

Quantities: active substances dosed at 4 10⁻⁸ g

Trace elements: magnesium, zinc, natrum

Plants: gentiana lutea (gentian)

Essential dietary factors: ilex paraguarientsis (maté), nasturtium officinale (cress), samphire, agaricus campester (cultivated psalliota mushroom), arachis hypogola (peanut), tricitum germinatius (germinated wheat), soja hispida (soya lecithin), piscis oleum (fish oils), chondrus crispus, porphyra umbilicalis, ostrea (oyster)

Vitamins: A, C, E, H Biotine, PP

Form of the product: capsules

### Example 7

### Circulation Function

Quantities: active substances dosed at 4 10⁻⁸ g

Trace elements: iron, magnesium, zinc

Plants: vitis vinifera (red vine)

Essential dietary factors: persea gratissima (avocado), ribes nigrum oleum acini (blackcurrant sedd oil), himanthalia elongata, porphyra umbilicalis, ostrea (oyster), enriched yeast

Vitamins: A, B1, B2, B5, B6, B9, C, E, F, PP

Form of the product: capsules

### Example 8

### Cell Regeneration Function

Quantities: active substances dosed at 4 10⁻⁸ g

Trace elements: zinc lactate, magnesium lactate, calcium lactate

Plants: rosa canina (rose hip), verbena officinalis (vervein), hibiscus abelmoschus

Essential dietary factors: zea mais (corn), coryllus avellana (hazelnut), solanum lycopersicum (tomato), daucus carot (carot), apium sativus (celery), oenothera biennis oleum (evening primrose oil), fucus vesiculosus, ostrea (oyster), yeast enriched with selenium, simple yeast

Vitamins: A, B1, B2 Riboflavine, B3=PP, B5, B6, C, E, H Biotine, B8

Form of the product: capsules

### Example 9

### Eyesight Function

Quantities: active substances dosed at 4 10⁻⁸ g

Trace elements: magnesium, zinc

Plants: ribes nigrum (blackcurrant)

Essential dietary factors: vaccinum myrtillus (blueberry), apium dulce (celery), brassica olcacera (cabbage), fagopyrum esculentum (buckwheat), nasturtum officinalis (cress), daucus carot (carot), porphyra umbilicalis, ostrea (oyster), enriched yeast

Vitamins: A, B1, B2, B5, B6, B9, C, E, F, H, PP

Form of the product: capsules

### Example 10

### Tonus Function

Quantities: active substances dosed at 4 10⁻⁸ g

Trace elements: iron, natrum, calcium, magnesium

Plants: rosa canina (rose hip), citrus aurantium (orange tree), mentha piperata (mint), hibiscus abelmoschus

Essential dietary factors: royal jelly, saccharomyces cerevisiae (brewer's yeast), agaricus campester (cultivated psalliota mushroom), ficus carica (fig), fagopyrum esculentum (buckwheat), nasturtum officinalis (cress), panax ginseng meyer (ginseng), palmaria palmata, porphyra umbilicalis

Vitamins: A, B1, B2, B5, B6, B9, C, E, F, H, PP

Form of the product: capsules

### Example 11

### Stress Metabolism

Quantities: active substances dosed at 4 10⁻⁸ g

Trace elements: magnesium, zinc, calcium

Plants: hibiscus abelmoschus, rosa canina (rose hip), citrus aurantium (orange tree), tilia europea (lime), verbena officinalis (vervein), anthemis nobilis (roman chamomile)

Essential dietary factors: citrus decumana (grapefruit), soja hispiga (soya), avocado, prunus amygdalus (almond), fragaria vesca (strawberry), amygdalus persica (peach), cerasus vulgarus (cherry), millium (millet), chondrus crispus, palmaria palmata

Vitamins: A, B1, B2, B3, B5, B9, B12, B15, C, E

Form of the product: capsules

### Example 12

### Regulation of Acupuncture Points

Quantities: active substances dosed at 10⁻⁸ g

Trace elements: copper, zinc, iron

Plants: matriarca chamomilla (chamomile), hypericum (St. John's-wort), arnica montana, amygdalus communis (almond), borrago officinalis (bourage), coriandre sativus (coriander), polygonatum vulgare (Sceau de Salomon)

Vitamins: B1, B2, B3, B5, B6, B8, C, E, F

Form of the product: gel for local application

### Example 13

### Detoxicating and Revitalizing Balsam

Quantities: active substances dosed at 10⁻⁸g

Trace elements: copper, zinc, sodium

Plants: arnica montana, viola tricolor (heartsease), amygdalus persica (peach tree), ribes nigrum (blackcurrant), bellis perennis (daisy), corylus avellana (hazelnut), lilum candidum (madonna lily)

Vitamins: A, B1, B2, B7, C, E, P, PP

Form of the product: gel for local application

### Example 14

### Buccal Function

Quantities: active substances dosed at 10⁻⁸ g

Trace elements: magnesium, copper, zinc

Plants: geum rivale (avens), eugenia caryophyllata (clove), sambucus nigra (elder), rosa gallica (red rose), vitis vinifera (red vine), ribes nigrum (blackcurrant), angelica archangelica (angelica), oenothera bienis (evening primrose)

Vitamins: A, B2, B5, C, PP

Form of the product: gel for local buccal application

### Example 15

### Regulation of Sugar and Fat Assimilation Metabolism

Trace elements: iron, zinc, magnesium

Plants: hibiscus, joe-pye weed, roman chamomile, jujube

Essential dietary factors: black radish, celery, chicory, borecole, pineapple, fucus, chondrus crispus, himanthalia elongata, green tea, orthosiphon, palm oil

Vitamins: A, B3, B6, C, E, H

Form of the product: capsules

### Example 16

### Fluid Balance

Trace elements: zinc, iron

Essential dietary factors: green tea, fennel, leek, parsley, pissenlit, vine, cherry, pineapple

Vitamins: B3, B6, C, E

Form of the product: drops

### Example 17

### Appetite Control

Trace elements: iron, zinc, magnesium

Plants: lime

Essential dietary factors: coriander, tarragon, garlic, wheat germ extract, apricot, papaya, grapefruit, dill, pineapple, celery, rosemary, chicory, brewer's yeast, caraway, green tea, redcurrant

Vitamins: B3, B6, H

Form of the product: tablets

### Example 18

### Sens of Taste Control

Essential dietary factors: coriander, garlic, dill, spring onion and chive, tarragon, pineapple, papaya, caraway seed, fennel

Vitamins: PP

Form of the product: salt

### Example 19

### Toxin Removal from Skin

Trace elements: magnesium, zinc, iron

Plants: roman chamomile, hibiscus

Essential dietary factors: palm oil, chicory, chondrus crispus, himanthalia, wheat germ, papaya, pineapple, selenium yeast

Vitamins: A, B1, B3/PP, B6, B9, C, E

Form of the product: liquid

### Example 20

### Skin Vitality

Trace elements: magnesium, iron

Plants: mint

Essential dietary factors: ginseng, royal jelly, brewer's yeast, organically-grown fig, organically-grown buckwheat, palmaria

Vitamins: A, B1, B3/PP, B6, B9, C, E, H, F

Form of the product: liquid

### Example 21

### Nutritional Balance of the Skin

Trace elements: magnesium, zinc

Plants: roman chamomile, hibiscus

Essential dietary factors: rosemary, grapefruit, papaya, pineapple, brewer's yeast

Vitamins: B1, B2, B5, B6, B9, C, F

Form of the product: liquid

### Example 22

### Relaxation of the Skin

Trace elements: sodium, magnesium

Plants: red vine

Essential dietary factors: blackcurrant seed oil, porphyra, cherry

Vitamins: A, B3/PP, C, E, H

Form of the product: liquid

### Example 23

### Overall Skin Beauty

Trace elements: magnesium, calcium

Plants: rose hip, orange tree, lime, vervein, roman chamomile

Essential dietary factors: chondrus, barley germ

Vitamins: A, B1, B2, B3/PP, B5, B9, B12, B15, C, E

Form of the product: liquid

1. A manufacturing method for products used in micronutrition, each of the products being constituted by one or more active substances in small quantities selected from the set consisting of the groups “trace element”, “plant”, “essential dietary factor”, “microorganism” or “vitamin”; said method being characterized in that it includes steps consisting in:
selecting from at least two of the groups of said set one or more active substances of each group according to the effects sought in the body,
combining in a sequential manner the active substances from each group by introducing each of them into an intermediate receptacle in the correct order and in the form of a liquid solution, and
combining them in a sequential fashion and in the pre-established order of the groups: “trace element”, “plant”, “essential dietary factor”, “microorganism”, and “vitamin”, the partial solutions being obtained by sequentially introducing each partial solution into a final receptacle after having dynamized the solution containing the partial solution(s) already contained in the final receptacle by shaking.

2. A manufacturing method for products used in micronutrition, each of the products being constituted by one or more active substances in small quantities selected from the set consisting of the groups “trace element”, “plant”, “essential dietary factor”, “microorganism” or “vitamin”; said method being characterized in that it includes steps consisting in:
selecting from at least two of the groups of said set one or more active substances from each group according to the effects sought in the body,
combining in a sequential manner the active substances from each group by sequentially introducing each of them in the form of a liquid solution into an intermediate receptacle, and
coating a plurality of granules by spraying droplets of each of said partial solutions in the pre-established order of groups “trace element”, “plant”, “essential dietary factor”, “microorganism” and “vitamin” so as to generate a final product in the form of granules, each of which carries a sequence of successive layers in said pre-established order.

3. The method according to claim 1 or 2 in which the step of obtaining said partial solution includes dynamization by shaking after the introduction of each solution of said active substance.

4. The method according to any of claims 1 through 3 in which each of said partial solutions is dynamized by shaking after the introduction of the last active substance.

5. The method according to any of claims 1 through 4 in which the quantity of said active substances is between 10⁻² g and 10⁻⁹ g per liter of solution

6. A micronutritional product obtained by implementation of the manufacturing method according to claims 1 through 5.

7. An application of the micronutritional product according to claim 6 to obtain a formulation intended for the prevention, self-regulation, nutrition and re-establishment of the body's physiological functions.

8. An application of the micronutritional product according to claim 6 to obtain a formulation intended for the prevention, care, re-equilibration and re-establishment of the body.

9. An application of the micronutritional product according to claim 6 to obtain a formulation intended for the nutrition and regulation of specific parts of the body and, in particular, acupuncture points.

10. An application of the micronutritional product according to claim 6 to obtain a formulation intended for local regeneration and revitalization (pain, contusions, burns, bites, etc.).

11. An application of the micronutritional product according to claim 6 to obtain a formulation intended for metabolic regulation.

12. An application of the micronutritional product according to claim 6 to obtain a formulation intended to help with the control and loss of weight.

13. An application of the micronutritional product according to claim 6 to obtain a formulation intended for skin care and vitalization.

14. An application of the micronutritional product according to claim 6 to obtain a formulation intended for buccal and dental care.
